(19) United States
(12) Patent Application Publication
(10) Pub. No.: US 2026/0157622 A1
(43) Pub. Date: Jun. 11, 2026

(54) METHOD AND SYSTEM FOR DETERMINING EYE TEST SCREEN DISTANCE

(71) Applicant: IBISVISION LTD., Glasgow (GB)

(72) Inventors: Peter Estibeiro, Glasgow (GB); William Silva, Glasgow (GB)

(73) Assignee: IBISVISION LTD., Glasgow (GB)

(21) Appl. No.: 18/706,480

(22) PCT Filed: Nov. 1, 2022
(86) PCT No.: PCT/EP2022/080436

§ 371 Date: May 1, 2024

(30) Foreign Application Priority Data
Nov. 1, 2021 (GB) 2115671.6
Nov. 1, 2021 (GB) 2115674.0
Nov. 1, 2021 (GB) 2115675.7

(51) Int. Cl. A61B 3/024; A61B 3/00; G06T 3/40; G06T 7/536

(52) U.S. Cl. CPC A61B 3/024; A61B 3/0041; G06T 3/40; G06T 7/536; G06T 2200/24; G06T 2207/30041; G06T 2207/30201

(57) ABSTRACT

A computer-implemented method of determining a distance from a display to a user's eye includes estimating, using a first technique, a distance between a display and a user's eye; and repeatedly estimating, using a second technique that is different to the first technique, a distance between the display and the user's eye.

### FIELD OF THE INVENTION

The present invention relates to measuring a distance between a user and a display. The invention has been developed primarily to assist in computer-based eye testing, and will be described with reference to that application. However, the skilled person will appreciate that the invention may be used in other applications. The invention further relates to a method and system for eye testing.

### BACKGROUND OF THE INVENTION

Eye testing typically involves having a user attempt to discern symbols or images on a chart. The symbols/images are generally presented in a controlled clinical setting, where factors such as a distance between the user and a chart are known.

### SUMMARY OF THE INVENTION

In accordance with a first aspect, there is provided a computer-implemented method for eye testing, comprising:
estimating a distance from a user to a display;
displaying one or more eye test images on the display, the eye test image(s) being displayed with an absolute dimension that is based at least in part on the estimated distance.

The method may comprise scaling the eye test image(s) to the absolute dimension based at least in part on a pixel density or pixel pitch of the display.

The absolute dimension may be an angular dimension.

The method may comprise:
repeatedly estimating the distance; and
scaling the eye test image(s) to maintain the absolute dimension responsive to the distance changing over time.

In accordance with a second aspect, there is provided a method of estimating a distance from a display to a user's eye, the method comprising: displaying, on a display, a first target;
displaying, on the display, a second target;
moving the second target relative to the first target;
receiving, via a user interface, input data from a user, the input data being indicative of the user having determined that the second target is no longer visible; and
estimating, based at least in part on a distance between the first target and the second target, a distance from the display to the user's eye.

Moving the second target relative to the first target may comprise holding the first target stationary on the display, and moving the second target.

Displaying the first target may comprise displaying an image and/or a video of a person.

The method may further include playing audio instructions regarding the user's interaction with the first and second targets.

The input data may be indicative of a user control input regarding the movement of the second target relative to the first target.

In accordance with a third aspect, there is provided a computer-implemented method of estimating a distance from a display to a user's eye, the method comprising:
estimating, using a first technique, a distance between a display and a user's eye;
repeatedly estimating, using a second technique that is different to the first technique, a distance between the display and the user's eye.

The first technique may comprise receiving, via a user interface, input data from a user to enable the distance to be estimated.

The first technique may comprise estimating the actual distance between the display and the user's eye.

The second technique may comprise determining a distance offset and adding it to, or subtracting it from, the distance estimated by the first technique.

The first technique may comprise:
displaying, on the display, a first target;
displaying, on the display, a second target;
moving the second target relative to the first target;
receiving, via a user interface, input data from a user, the input data being indicative of the user having determined that the second target is no longer visible; and
determining, based at least in part on a distance between the first target and the second target, the distance from the display to the user's eye.

Moving the second target relative to the first target may comprise holding the first target stationary on the display, and moving the second target.

Displaying the first target may comprise displaying an image and/or a video of a person.

The method may include playing audio instructions regarding the user's interaction with the first and second targets.

The input data may be indicative of a user control input regarding the movement of the second target relative to the first target.

The method may comprise receiving further input data from a further user, wherein moving of the second target relative to the first target is performed at least partly based on the further input data.

The second technique may comprise automatically estimating the distance between the display and the user's eye without user input.

The second technique may comprise repeatedly capturing an image of at least a portion of the user's face, and determining the distance offset based on a change in image size related to one or more features within the portion of the user's face.

The portion may comprise at least the user's eyes, and the change in image size comprises a distance between portions of the user's eyes.

In accordance with a fourth aspect, there is provided a method of adjusting a size of one or more images on a display using the method according to the third aspect, the method of adjusting the size comprising:
displaying at least one image on the display, an onscreen dimension of the image being at least partly based on the distance estimated using the first technique;
scaling the onscreen dimension of the image at least partly based on the distance estimated using the second technique.

The onscreen dimension may be based on an angular viewing dimension.

Scaling the onscreen dimension may comprise maintaining an angular viewing dimension of the image for the user, as the distance changes over time.

In accordance with a fifth aspect, there is provided a data processing system comprising means for carrying out the method of any preceding aspect.

In accordance with a sixth aspect, there is provided a computer program comprising instructions that, when the program is executed by a computer, cause the computer to carry out the method of any aspect.

### BRIEF DESCRIPTION OF THE DRAWINGS

Aspects and implementations will now be described, without limitation and by way of example only, with reference to the accompanying drawings, in which:

FIG. 1 is a schematic side view of a user viewing a display;

FIG. 2 is a schematic of a computer system;

FIG. 3 is a flowchart showing a method of displaying one or more eye tests on a display;

FIG. 4 is a sequence of user views of the display of FIG. 1;

FIG. 5 is a flowchart showing a method of estimating a distance between the display user;

FIG. 6 is a front view of a scaling template placed over a display;

FIG. 7 is a schematic plan view of a user's eye and a display;

FIG. 8 is a flowchart showing a method of estimating a distance between a display and a user; and

FIGS. 9 and 10 are schematic views of a user's face.

### DETAILED DESCRIPTION OF THE INVENTION

The present disclosure relates to the interaction of a user with a display, such as a computer display. The disclosure describes various systems and methods in which a distance between a user (and a user's eye, in particular) and such a display can be estimated, and in which images to be displayed on the display can be scaled based on the estimated distance. The words “estimated” and “determined”, and related words, are used interchangeably throughout this application.

The present disclosure has been developed in the context of optometric testing, and will be described with reference to that application. Many conventional methods for examining vision rely on the patient's subjective responses to images presented to them. The size and separation of elements within the images is designed to assess the patient's ability to distinguish the spatial separation between objects based on angular units of arc subtended from the eye to the image.

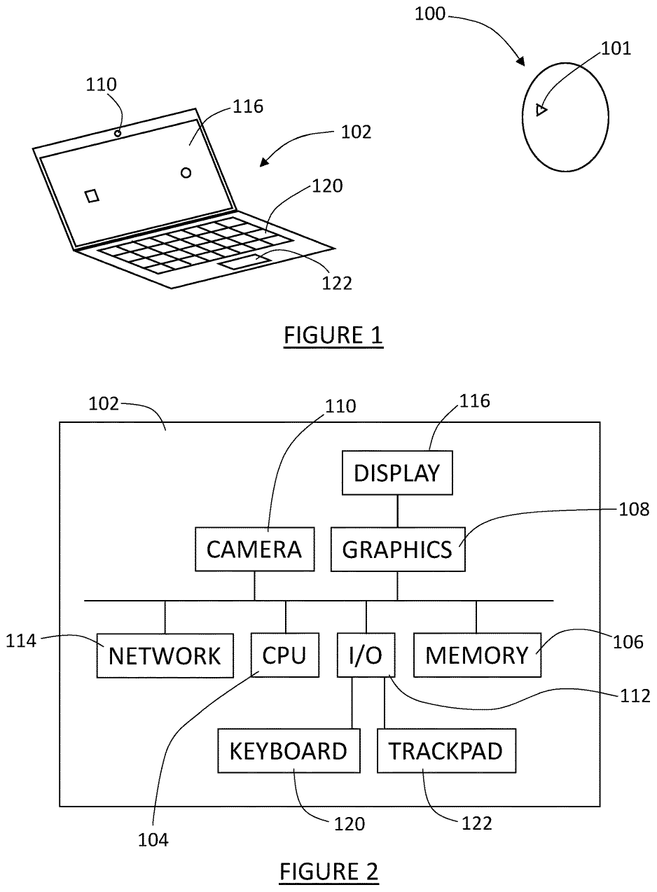

Referring to the drawings, FIG. 1 shows a user 100 having an eye 101, and a system in the form of a laptop 102. User 100 is positioned a suitable distance from laptop 102, and may be given instructions about a suitable range of distances for the circumstances. For example, a typical range of such distances for a medium size laptop is about 0.3 m to 0.9 m (approximately 1′ to 3′), dependent upon factors such as the size of the display, and the particular tests to be performed. For example, for a larger display, user 100 may be instructed to position themselves further away.

For a distance visual acuity test, user 100 may need to be more distant from the display, such as more than about 3 m (about 10′) from the display. As best shown in FIG. 2, laptop 102 can include a processor in the form of a CPU 104, memory 106 (including volatile memory such as DRAM and non-volatile memory such as a solid-state hard-drive), a graphics processor 108, an image capture device in the form of a camera 110, an I/O system 112, and a network interface 114 all connected to each other by one or more bus systems and connectors, represented generally in FIG. 2 as a bus 118.

Graphics processor 108 outputs graphics data for display on a display 116.

I/O system 112 accepts user inputs from a keyboard 120 and a trackpad 122.

Memory 106 stores software including an operating system and one or more computer software programs. CPU 104 is configured to execute the operating system and computer programs stored by memory 106. The computer program(s) stored by memory 106 include instructions for implementing any and all of the methods described in the current application.

Camera 110 captures still images and video, including still images and video of user 100 in certain circumstances, as described in more detail below.

Network interface 114 is configured to communicate through a network via a switch, router, wireless hub, or telecommunications network (not shown), optionally including the Internet.

The hardware of laptop 102 is conventional, and so is not described in further detail.

The skilled person will appreciate that systems for implementing the described aspects can take any suitable form, including integrated systems in which all hardware forms part of a single device such as a mobile telephone or laptop, or a distributed system, where at least some individual components form part of different devices. For example, the display can take the form of a television, mobile phone, computer tablet, or computer monitor. Optionally, the display can be a touchscreen, which accepts user input in addition to (or instead of) keyboard 120 and trackpad 122. Any required user input can be obtained via the device that displays the images, or via any other suitable input device. For example, a mobile telephone may be used to accept user input, while images are displayed on an Internet-connected television receiving image data remotely via the Internet or a local area network, or by casting from the mobile telephone. Some or all of the computer software instructions can be stored and/or run on a remote computer such as a server.

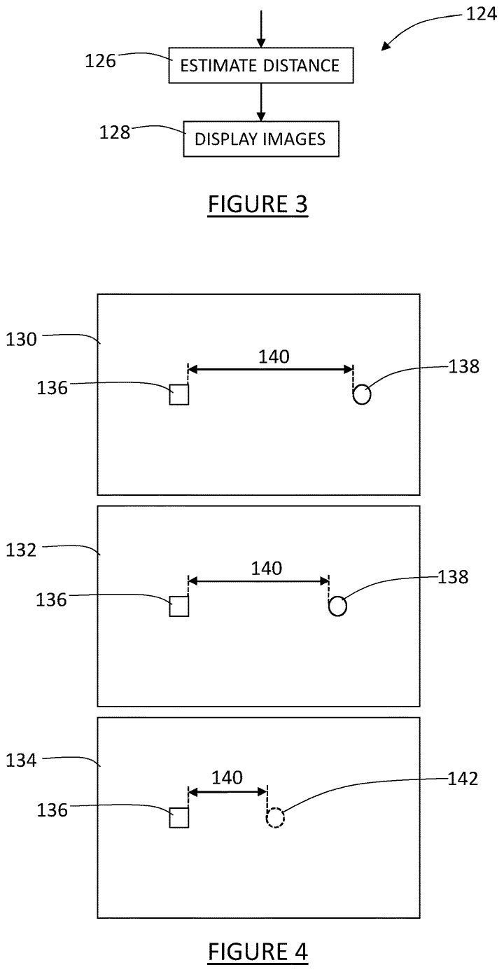

Turning to FIG. 3, there is disclosed a computer-implemented method 124. Method 124 comprises estimating 126 a distance from user 100 to display 102, as described in more detail below.

Any suitable method may be used to estimate 102 the distance from user 100 to display 116. For example, user 100 may be instructed to use a ruler or measuring tape to measure the distance from eye 101 to the display, or such measurements may be made by an optometrist or someone assisting user 100. Alternatively, camera 110 may be used to capture an image of user 100, and a distance estimated based on a distance between facial landmarks, such as the eyes, as compared with average distances between such landmarks in the general population.

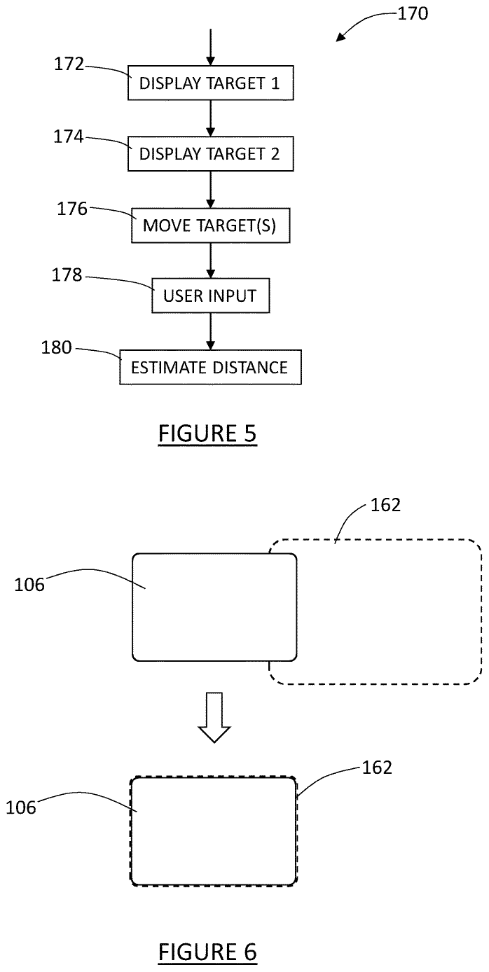

One method 170 for estimating the distance from user 100 to display 116 will be described with reference to FIGS. 4 and 5. FIG. 4 shows a sequence of user views 130, 132 and 134, showing what user 100 perceives to be displayed on display 116 of laptop 102. FIG. 5 shows method 170 for estimating the distance from user 100 to display 116.

A user is instructed to sit with their face about 500 mm from display 116. A first target in the form of a square 136 is displayed 172 on display 116, and a second target in the form of a circle 138 is displayed 174 on display 116.

Based on the approximately 500 mm initial distance, square 136 can be presented at a size that subtends up to approximately 5×5 degrees (=about 44×44 mm at 500 mm user-display distance) of the user's visual field, although a higher subtended angle may be acceptable in some circumstances.

On the same basis, circle 138 can be sized to be Goldmann III 4e as perceived by the user. As is understood by the skilled person, Goldmann III is 0.43 degrees of arc, which equates to 4 mm at a 500 mm observation distance, and 4e indicates maximum contrast/brightness.

One specific alternative size for circle 138 is Goldmann V, which is about 15 mm at a 500 mm observation distance, although any other suitable size may be employed to suit a particular implementation.

The colours and contrast of the first and second targets can be selected by the skilled person to achieve desired outcomes. For example, high contrast colours/shades (e.g., black against a white background) may provide relatively good results for the second target. The use of bright and/or primary colours for the first target, or the background to the first target, may be more engaging for some users, such as younger users whose concentration levels may be lower than a typical adult's.

Other colour combinations may be used. For example, blue and yellow may be a useful contrasting combination for certain eye conditions.

Although the first target is described as a square and the second target is described as a circle, it will be appreciated that either or both targets may take different forms. For example, the first target may take the form of a brief instruction, such as “FOCUS” or “LOOK HERE” (e.g., with “LOOK” positioned above “HERE”), optionally with an arrow or other indicator drawing the user's attention to a focal point.

Alternatively, the first target may take the form of a compact or thumbnail video, which may be a live video of an optometrist or an assistant, or a pre-recorded video, for example giving the user instructions. Such a video may be presented at a size that subtends up to, for example, 5×5 degrees of the user's visual field, although a higher subtended angle may be acceptable in some circumstances.

The video may also take the form of a cartoon or other stylized representation, including a cartoon or stylized representation of an optometrist or an assistant, or a friendly animal, well-known character, mascot, avatar, or the like. Such stylization can be performed with video filtering, either live or in post-production. Stylization may assist with anonymization of the optometrist or an assistant, and may also allow for optimization of the video image to maximize contrast, for example. Some patients, and particularly children, may find it easier to focus on an animated target than a static image. Maintaining focus may be of particular importance when, for example, mapping a patient's visual field or defining scotomas including the naturally occurring blind spot, as well as areas of defective vision resulting from disease, trauma, etc.

Similarly, the second target may take any other suitable form. However, because the second target is intended to be in the user's peripheral vision, it may be less desirable to use, for example, videos or more complex images, as compared with the first target. Nevertheless, the size, color, and other aspects of the second target may be selected, in conjunction with the display background colour, to maximize the likelihood of accurate measurement.

Returning to FIG. 4, an initial distance 140 between square 136 and circle 138 can be selected to ensure that the angle sub-tended by them at the user's eye 101 is more than the angle sub-tended by average fovea and blind spot spacing in the human eye. The spacing can, for example, be selected based on an assumed maximum distance between the user and the screen based on the instructions given to the user, or can be based on a rough initial estimate based on spacing between the user's facial landmarks as captured by camera 110, for example.

In view 130, square 136 is positioned to the left of centre, and circle 138 is positioned close to the right-hand edge of display 116. There may optionally be a small vertical offset between square 136 and circle 138, to at least partly account for the approximately 1.5 degree vertical offset between the human fovea and retinal blind spot.

User 100 is instructed to cover or close their left eye and to focus their right eye 101 on square 136. These instructions (and those that follow) can be provided by the software in the form of an on-screen message, and/or in audio form, such as by way of a recorded or synthesized voice. Alternatively, an optometrist or assistant can provide the user with instructions as the test proceeds.

User 100 is instructed to note when circle 138 apparently disappears from their peripheral field as circle 138 moves relative to square 136.

Circle 138 is then moved 176 on display 116 relative to square 136.

Relative movement between circle 138 and square 136 may be achieved in any suitable manner. For example, square 136 may be kept stationary on display 116, while circle 138 is moved. Alternatively, circle 138 may be kept stationary on display 116, while square 136 is moved. In yet other alternatives, both square 136 and circle 138 may be moved on display 116 such that the relative distance between them changes. The relative movement may be generally linear, with square 136, circle 138, or both, moving generally towards each other. Where a vertical offset is used to at least partly account for the 1.5 degree vertical offset of the retinal blind spot of an average human eye relative to the horizon, the angle at which square 136 and circle 138 move relative to each other may at least partly take that offset into account.

The relative movement may be controlled in any suitable manner. For example, user 100 may be instructed to use keyboard 120 or trackpad 122 to control the relative movement at a rate that they find comfortable. Alternatively, the relative movement may be automatically controlled by the software running on laptop 102. In yet other alternatives, an optometrist or assistant may control the relative movement, with the optometrist or assistant optionally being situated remote from user 100 and controlling the relative movement remotely via a network such as the Internet and/or a local area network.

In the sequence of views 130 to 134 shown in FIG. 4, square 136 is held stationary and circle 138 is moved towards square 136.

In first view 130, circle 138 and square 136 are displayed on display 116 horizontally spaced apart from each other.

In second view 132, circle 138 has moved towards square 136. Distance 140 between circle 138 and square 136 is reduced compared to view 130, but circle 138 is still visible in the peripheral vision of user 100.

In third view 134, circle 138 has moved to a position 142 in which it is no longer visible to user 100. This is because position 142 is over the user's retinal blind spot (the position within the retina of a human eye where the optic nerve passes through a rear wall of the eyeball, and where there are no photoreceptors).

When user 100 observes that circle 138 is no longer visible in their peripheral vision, they input 178 this information through a user interface such as keyboard 120, trackpad 122, or any other suitable input mechanism. For example, user 100 may be instructed to left-click on trackpad 122, press the space bar on keyboard 120, or speak an instruction such as the word “stop” into a microphone (not shown) when circle 138 disappears.

Whichever form it takes, this user input is interpreted as being indicative of the user having determined that circle 138 is no longer visible in the user's peripheral field.

Optionally, further rounds of movement and user feedback may be performed, in order to potentially improve accuracy of the position. For example, the user may be instructed to indicate both when the second target disappears, and then when it reappears as the second target leaves the retinal blind spot. An average of the distances of disappearance and reappearance can be taken, which may provide greater accuracy at the cost of additional complexity for the user. Alternatively, or in addition, the measurement process may be repeated, and the measured distances averaged. Alternatively, or in addition, any or all of these procedures can be performed on both eyes and an average taken.

Once distance 140, when circle 138 is at position 142 as shown in view 134 of FIG. 4, has been determined, a distance from display 116 to the user's eye 101 can be estimated 180 using that distance and the average angular position of the human retinal blind spot.

Alternatively, the first and second targets can initially be positioned relatively close to each other or even wholly or partly superimposed, where they will both be visible to the user. The first and second targets (square 136 and circle 138, for example) are then moved away from each other until the second target disappears from the user's peripheral vision. The rest of the process may be as described above.

Alternatively, the second target may be displayed intermittently. For example, it may be presented at a first position, removed from that first position (i.e., not displayed at all) for a period of time, and then presented again at a second position that is spaced from the first position. The process is repeated for further positions, each at a different position relative to the first target. The user may be instructed to indicate via the user interface each time they see the second target appear. When the system notes that the user does not react to the appearance of the second target, it may be inferred that the second target is within the retinal blind spot. As described earlier, differences in the distance between the first and second targets are relative, and the first target may also move, optionally while the second target is not displayed.

A potential complicating factor is the relationship between display resolution (measured in pixels) and pixel pitch (the spacing, typically measured in micrometres, between pixels). (Note: although “pixel pitch” is referred to herein, this concept may be interchanged with “pixel density”. Pixel pitch and pixel density are inverses of each other). For example, commonly used 1080 high definition formats are 1920 pixels wide and 1080 pixels high. However, this resolution is employed on a range of display sizes, on devices such as mobile telephones, tablets, computer displays, and large screen televisions. Without knowing the pixel pitch, it is not possible to know the physical linear size (i.e., as measured on the surface of display 116) of an image displayed on a particular display. Accordingly, it is necessary to determine the display's pixel pitch or pixel density, either directly, or based on a relationship between, for example, the display's resolution and physical dimensions.

There are several ways in which this information may be determined. For example, software running on laptop 102 may be able to access information about the display screen's parameters from the operating system. For example, the operating system may enable software to look up the pixel pitch/density, or the resolution and physical dimensions of the display, by way of an API.

Different hardware and software systems may offer access to different parameters. For example, some operating systems may enable software to access resolution information, but not the physical dimensions of an associated display, or the pixel pitch/density. This may particularly be the case where the display is a separate piece of hardware, about which the operating system may not know details beyond its resolution (which is required in most systems to enable the display to be appropriately driven). However, even with laptops, where the display is built-in, the operating system may not be able to supply information about the pixel pitch/density and/or physical display dimensions.

Where a display's resolution information can be accessed via, for example, an API, but pixel pitch/density and/or physical dimensions are not accessible, there are other ways in which the pixel pitch/density and/or physical dimensions of the display may be determined. For example, a user may be instructed to input manufacturer and model details, and/or a serial number, from the display, which can be used to look up the display's dimensions online. This can be done automatically by software, or manually by a user following instructions provided by the software or its documentation.

Alternatively, a user may be asked to measure the display's dimensions, including its horizontal and vertical dimensions, and/or its diagonal dimension, and input them to the laptop via the keyboard and/or trackpad, for example.

Alternatively, a physical template or calibration element may be used in conjunction with the software to determine the required information, potentially without knowing the display's resolution or physical dimensions. An example of such a template is shown in FIG. 6, which shows a template 106. For user convenience, template 106 may take the form of a credit card, which come in a standard size of 3.37″×2.125″ (85.6 mm×53.98 mm).

As shown in FIG. 6, template 106 is held against display 116 while a rectangle 162 having the same aspect ratio as template 106 is displayed on display 116. The user can adjust, via a user interface such as keyboard 120 and/or trackpad 122, a size and position of rectangle 162 until rectangle 162 is just visible around all edges of template 106. The user then indicates via the user interface that the resizing and repositioning is complete. The pixel pitch can then be determined based on the relationship between display resolution (known from the operating system) and the size of displayed rectangle 162 needed to correspond with the known size of template 106.

As an example, for a 19″ computer monitor with a resolution of 1920×1080, rectangle 162 will measure 391×248 pixels, giving a pixel density of 391/85.6=4.57 pixels/mm. As mentioned above, pixel density is the inverse of pixel pitch, and the skilled person will understand that references to one within the current application can be considered references to the other with the required inverse operation applied.

Although a rectangle 162 is shown, other forms of onscreen images may be displayed for interaction with template 106. For example, a pair of horizontally opposed arrows and a pair of vertically opposed arrows may be displayed, with each pair of arrows being spaced apart from each other and pointing inwards. Template 106 is placed against display 116 in the space between the arrow points. The position of the arrows, and the distance between arrow points, is controlled by the user by way of the user interface until all arrow points are visible outside the template edge. Pixel pitch can then be determined as described above.

Other shapes, sizes and forms of templates may be used in different implementations.

Once the pixel pitch and/or display dimension parameters have been acquired, the system can use that information along with the known display resolution and the determined distance between the circle and square to estimate a distance from display 116 to the user's eye 101.

The number of pixels between square 136 and circle 138 can be converted to a linear distance based on the pixel pitch (which can be determined, if needed, based on the display's resolution and physical dimensions). For example, if the distance is 400 pixels and the pixel pitch is 250 micrometres, the distance is 400×250=100 mm.

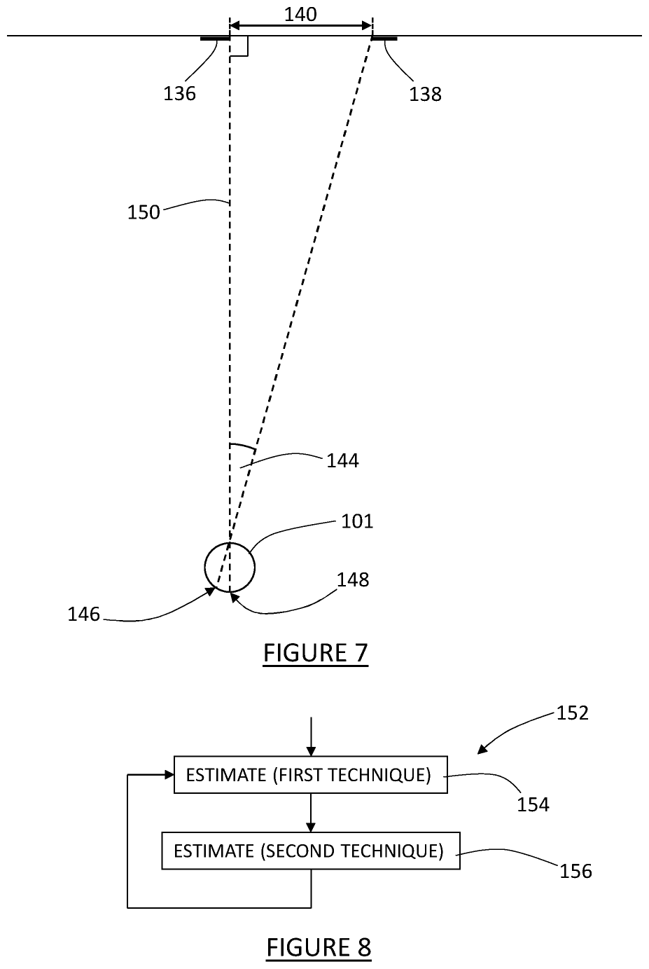

As shown in FIG. 7, which is a schematic plan view of user's eye 101 and display 116 (not to scale), an angle 144 subtended by a retinal blind spot 146 and a fovea 148 of eye 101 is approximately 12.5 to 15 degrees temporal and 1.5 degrees below the horizon for an average human.

Based on the retinal blind spot angle and the (linear) distance 140 shown in third view 134 of FIG. 4, trigonometry can be used to estimate a distance 150 between user's eye 101 and display 116. For example, if distance 140 is 100 mm, and the average fovea to blind spot angle is taken as the average of 12.5 and 15 degrees (=13.75 degrees), then the following equation may be used to determine distance 150:

$$tan ( 1375 ) = 100 / distance$$

$$distance = 100 / tan ( 1375 )$$

$$distance = 408.7 mm$$

The skilled person will appreciate that FIG. 7 assumes that eye 101 is aligned with the right-hand edge of square 136. The user can be instructed to align their eye in this way, or alternatively can be instructed to position it directly in line with the centre of square 136. Whichever position is selected, the details of the equation and the values used can be adjusted in a manner known to those skilled in the art, in order to ensure that a desired accuracy is achieved.

The skilled person will also appreciate that although a relatively simple trigonometric approach has been described above, different trigonometric, geometric and/or other types of equation(s) may be employed. For example, such equations may more precisely model the optical system of the eye, including the convergent functionality of the cornea and lens, the curve of the retina, and the flat nature of the display.

Returning to FIG. 3, once distance 150 from display 116 to user's eye 101 has been estimated 126, one or more test images are displayed 128 on display 116, the eye test image(s) being presented with an absolute dimension that is based at least in part on the determined distance. The test images may take the form of, for example, images sized in accordance with the crowded logMAR scale, as will be understood by the skilled person.

In this context, “absolute dimension” refers to a physical size of the images on the display. This physical size can be measured in any suitable manner. For example, especially when performing optometric testing, it is important that the user be presented with images having a known and controllable angular dimension. For example, part of an optometric test may require the presentation of a row of letters to a user, each letter subtending, say, 5 minutes of arc (5/60 degrees) in the vertical plane. An alternative way of expressing this is by the displayed height/width and user-display distance. For example, 2 degrees at 500 mm distance equates to 17.46 mm high at 500 mm distance, or 26.19 mm high at 750 mm distance.

It is not possible to directly output an image having the desired subtended angle, since the angle will change depending upon a distance from user 100 to display 116. However, once the distance between user 100 and display 116 has been determined in step 126, it is possible to appropriately scale the images to be displayed such that they subtend the desired angle. Such scaling can be performed based at least in part on the pixel pitch or pixel density of the display. A difficulty that may be faced in practice is that users may move, consciously or sub-consciously, to get more comfortable during an optometric test. If the user moves towards or away from the display, the angular dimensions of the images on the screen relative to the patient change, and the results of the examination may be less reliable. There is also a natural human tendency to move closer to an object in order to see it more clearly.

It is possible to reduce the impact of a user moving towards or away from display 116 by repeatedly estimating the distance, and scaling the eye test image(s) to maintain the absolute dimension responsive to the distance changing over time. While it is possible to use the retinal blind spot method described above for these additional distance estimates, that method requires the user to focus their eyes on target images. This requires the optometric test to be paused while the distance is established, which may be disruptive and time-consuming. Moreover, user movements may take place over a relatively short period of time and may not be captured by periodic (say, tens of seconds or more) application of the retinal blind spot distance estimation method.

Referring to FIG. 8, there is shown a method 152 of estimating a distance from a display to a user's eye. Method 152 includes estimating 154, using a first technique, a distance between a display and a user's eye, and then repeatedly estimating 156, using a second technique that is different to first technique 154, a distance between display 116 and user's eye 101.

The first technique may comprise receiving, via a user interface, input data from a user to enable the distance to be estimated. That is, the first technique may require user interaction, and may therefore potentially disrupt or prevent the user from performing other activities, such as interacting with optometric images as part of an optometric test. However, if the first technique is performed before optometric testing commences, or only relatively infrequently during such optometric testing, the disruption may be minimized or even avoided.

The first technique may take the form of the retinal blind spot distance estimation method described above. Alternatively, the first technique may take the form of any other suitable method for estimating a distance between a user and a display. For example, user 100 may be instructed to use a ruler or measuring tape to measure the distance from their eye to the display, or such measurements may be made by an optometrist or someone assisting user 100. Preferably, the first technique involves estimating an actual distance, rather than a relative distance.

The second technique may be, for example, a technique that does not require the user to interrupt the optometric testing process. For example, the second technique can comprise automatically estimating the distance between the display and the user's eye without user input.

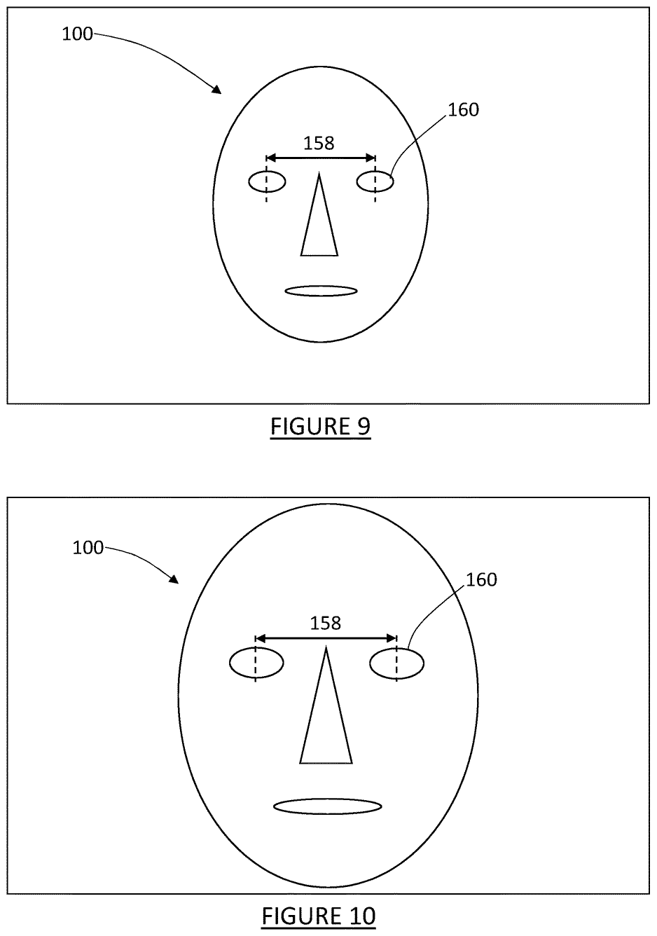

One method of estimating the distance between the display and the user's eye without user input will be described with reference to FIGS. 9 and 10, which show schematic front views of user 100 captured by camera 110. The image captured in FIG. 9 is captured immediately or at least shortly after the first technique has been used to determine the distance between user 100 and display 116. A distance 158 between the eyes 160 of user 100 is determined. The skilled person will appreciate that this can be achieved in any of a number of ways. For example, a number of pixels between the centres of eyes 160 may be determined (i.e., as shown in FIGS. 9 and 10). Alternatively, a number of pixels between the edges of the eyes, or between some combination of eyes, nose, mouth, or other facial landmarks may be determined, or a change in size of a facial feature or features may be determined. Facial feature identification and tracking is known to the skilled person, and so is not described in further detail.

It is not strictly necessary for the second technique to determine an actual distance between whatever facial landmarks are identified, because subsequent steps in the second technique determine only a relative change in the distance between user 100 and display 160.

Turning to FIG. 10, a further image is captured, in which user 100 has moved closer to camera 110. The effect of this movement is for the user's eyes 160 to move further apart. Distance 158 between eyes 160 is again determined, and then a ratio of the distance in FIGS. 9 and 10 is determined.

By repeatedly determining a ratio of the change in distance between facial landmarks between sequential image captures (or images within a video), the distance can be continuously updated. The rate at which the distance is updated can be chosen to suit the implementation.

The rate at which the displayed images are scaled based on the user's movement may not necessarily match the rate at which any change in distance is determined. For example, relatively small changes in distance may be ignored, especially if they will not unduly affect the results of the test being undertaken.

To encourage a user to stay still, reminders may be provided, either onscreen or via audible instructions. Such instructions may be periodic, and/or may be based on the system noting that the user has moved beyond a certain tolerance.

If the images are to be scaled based on a change in user position, the user may optionally be warned in advance, and optionally the test may be paused while the images are scaled.

Although the use of facial landmarks has been described, the skilled person will appreciate that other markers may be used, such as one or more physical markers, or one or more biometric markers associated with the patient. Examples of usable markers include spectacle frames, stickers on the face or elsewhere, or biometric markers or coordinates such as distances between features like eyes, nose, ears, chin, etc., that are used for facial recognition.

Although it may be convenient for an image capture device such as camera 110 to be in the same focal plane as display 116, in practice it is not limited to this location as long as its location relative to the display screen and the patient is known.

The sequence of distances that are determined by the application of the first and second techniques are applied to scaling an image displayed on display 116. For example, as part of an optometric test, at least one image may be displayed on display 116. An onscreen dimension of the image is at least partly based on the distance estimated using the first technique.

The second technique is then performed, and any change in distance between user 100 and display 116 is used to scale the onscreen dimensions of the image. As described above, the onscreen dimension may be based on an angular viewing dimension, which may be kept constant, for example, as the user moves relative to display 116. For example, as the user moves closer to display 116, the image will be made smaller so that it maintains the same angular dimensions.

The scaling may be applied automatically by the software, or manually by an operator.

Instead of, or in addition to, scaling the size of one or more optometric images, a separation between two or more images on the display may be scaled in response to the changes in the user's distance from the display. Such separation scaling can be applied particularly to visual field analysis and to the superposition of examination images onto a background video.

Allowing a patient to sit in a comfortable position and then automatically scaling on-screen images to account for any changes in display-patient distance provides relatively good results, especially where the patient is not in a clinical setting where light restraints, forehead or chin rests, and the like can be used to ensure an accurate and consistent display-patient distance. The fact that the patient can to an extent choose where they position themselves helps with patient comfort and compliance. Accordingly, although the invention can be applied in a clinical setting, it may also be applied in a telemedical manner, allowing automated or clinician-led testing to be performed while the patient is at home or in another non-clinical setting.

Although the invention has been described with reference to a number of aspects, examples and alternatives, the skilled person will appreciate that the invention may be embodied in many other forms.

1. A computer-implemented method of determining a distance from a display to a user's eye, the method comprising:
estimating, using a first technique, a distance between a display and a user's eye;
repeatedly estimating, using a second technique that is different to the first technique, a distance between the display and the user's eye.

2. The method as claimed in claim 1, wherein the first technique comprises receiving, via a user interface, input data from a user to enable the distance to be estimated.

3. The method as claimed in claim 1 or claim 2, wherein the first technique comprises estimating the actual distance between the display and the user's eye.

4. The method as claimed in any one of the preceding claims, wherein the second technique comprises determining a distance offset and adding it to, or subtracting it from, the distance estimated by the first technique.

5. The method as claimed in any one of the preceding claims, wherein the first technique comprises:
displaying, on the display, a first target;
displaying, on the display, a second target;
moving the second target relative to the first target;
receiving, via a user interface, input data from a user, the input data being indicative of the user having determined that the second target is no longer visible; and
determining, based at least in part on a distance between the first target and the second target, the distance from the display to the user's eye.

6. The method as claimed in claim 5, wherein moving the second target relative to the first target comprises holding the first target stationary on the display, and moving the second target.

7. The method as claimed in claim 5 or claim 6, wherein displaying the first target comprises displaying an image of a person.

8. The method as claimed in any one of claims 5 to 7, wherein displaying the first target comprises displaying a video of a person.

9. The method as claimed in any one of claims 5 to 8, further including playing audio instructions regarding the user's interaction with the first and second targets.

10. The method as claimed in any one of claims 5 to 9, wherein the input data is indicative of a user control input regarding the movement of the second target relative to the first target.

11. The method as claimed in any one of claims 5 to 10, comprising receiving further input data from a further user, wherein moving of the second target relative to the first target is performed at least partly based on the further input data.

12. The method as claimed in any one of the preceding claims, wherein the second technique comprises automatically estimating the distance between the display and the user's eye without user input.

13. The method as claimed in any one of the preceding claims, wherein the second technique comprises repeatedly capturing an image of at least a portion of the user's face, and determining the distance offset based on a change in image size related to one or more features within the portion of the user's face.

14. The method as claimed in claim 13, wherein the portion comprises at least the user's eyes, and the change in image size comprises a distance between portions of the user's eyes.

15. A method of adjusting a size of one or more images on a display using the method of any one of the preceding claims, the method of adjusting the size comprising:
displaying at least one image on the display, an onscreen dimension of the image being at least partly based on the distance estimated using the first technique;
scaling the onscreen dimension of the image at least partly based on the distance estimated using the second technique.

16. The method as claimed in claim 15, wherein the onscreen dimension is based on an angular viewing dimension.

17. The method as claimed in claim 16, wherein scaling the onscreen dimension comprises maintaining an angular viewing dimension of the image for the user, as the distance changes over time.

18. A data processing system comprising means for carrying out the method of any one of the preceding claims.

19. A computer program comprising instructions that, when the program is executed by a computer, cause the computer to carry out the method of any one of claims 1 to 17.

20. A method of determining a distance from a display to a user's eye, the method comprising:
displaying, on a display, a first target;
displaying, on the display, a second target;
moving the second target relative to the first target;
receiving, via a user interface, input data from a user, the input data being indicative of the user having determined that the second target is no longer visible; and
determining, based at least in part on a distance between the first target and the second target, a distance from the display to the user's eye.

21. The method as claimed in claim 20, wherein moving the second target relative to the first target comprises holding the first target stationary on the display, and moving the second target.

22. The method as claimed in claim 20 or claim 21, wherein displaying the first target comprises displaying an image of a person.

23. The method as claimed in any one of claims 20 to 22, wherein displaying the first target comprises displaying a video of a person.

24. The method as claimed in any one of claims 20 to 23, further including playing audio instructions regarding the user's interaction with the first and second targets.

25. The method as claimed in any one of claims 20 to 24, wherein the input data is indicative of a user control input regarding the movement of the second target relative to the first target.

26. The method as claimed in any one of claims 20 to 25, comprising receiving further input data from a further user, wherein moving of the second target relative to the first target is performed at least partly based on the further input data.

27. A data processing system comprising means for carrying out the method of any one of claims 20 to 26.

28. A computer program comprising instructions that, when the program is executed by a computer, cause the computer to carry out the method of any one of claims 20 to 26.

29. A computer-implemented method for eye testing, comprising:
estimating a distance from a user to a display;
displaying one or more eye test images on the display, the eye test image(s) being displayed with an absolute dimension that is based at least in part on the estimated distance.

30. The method as claimed in claim 29, comprising scaling the eye test image(s) to the absolute dimension based at least in part on a pixel density or pixel pitch of the display.

31. The method as claimed in claim 29 or claim 30, wherein the absolute dimension is an angular dimension.

32. The method as claimed in claim 31, comprising:
repeatedly estimating the distance; and
scaling the eye test image(s) to maintain the absolute dimension responsive to the distance changing over time.

33. A data processing system comprising means for carrying out the method of any one of claims 29 to 32.

34. A computer program comprising instructions that, when the program is executed by a computer, cause the computer to carry out the method of any one of claims 29 to 32.
